SEQUENCE LISTING (6 sequences)

SEQ ID NO: 1 - DNA, 1061 bp, Homo sapiens
caggaggatggaggcccacaacgcgtctgccccattcaacttcaccctgccacccaactttggcaagcgccccacagacctggcactgagcgtcatcctggtgttcatgttgttcttcatcatgctctcgctgggctgcaccatggagttcagcaagatcaaggctcacttatggaagcctaaagggctggccatcgccctggtggcacagtatggcatcatgcccctcacggcctttgtgctgggcaaggtcttccggctgaagaacattgaggcactggccatcttggtctgtggctgctcacctggagggaacctgtccaatgtcttcagtctggccatgaagggggacatgaacctcagcattgtgatgaccacctgctccaccttctgtgcccttggcatgatgcctctcctcctgtacatctactccagggggatctatgatggggacctgaaggacaaggtgccctataaaggcatcgtgatatcactggtcctggttctcattccttgcaccatagggatcgtcctcaaatctaaacggccacaatacatgcgctatgtcatcaagggagggatgatcatcattctcttgtgcagtgtggccgtcacagttctctctgccatcaatgtggggaagagcatcatgtttgccatgacaccactcttgattgccacctcctccctgatgccttctattggctttctgctgggttatgttctctctgctctcttctgcctcaatggacggtgcagacgcactgtcagcatggagactggatgccaaaatgtccaactctgttccaccatcctcaatgtggcctttccacctgaagtcattggaccacttttcttctttcccctcctctacatgattttccagcttggagaagggcttctcctcattgccatattttggtgctatgagaaattcaagactcccaaggataaaacaaaaatgatctacacagctgccacaactgaagaaacaattccaggagctctgggaaatggcacctacaaaggggaggactgctccccttgcacagcctagccct

SEQ ID NO: 2 - DNA, 3966 bp, Homo sapiens
atgtctgactcagtaattcttcgaagtataaagaaatttggagaggagaatgatggttttgagtcagataaatcatataataatgataagaaatcaaggttacaagatgagaagaaaggtgatggcgttagagttggcttctttcaattgtttcggttttcttcatcaactgacatttggctgatgtttgtgggaagtttgtgtgcatttctccatggaatagcccagccaggcgtgctactcatttttggcacaatgacagatgtttttattgactacgacgttgagttacaagaactccagattccaggaaaagcatgtgtgaataacaccattgtatggactaacagttccctcaaccagaacatgacaaatggaacacgttgtgggttgctgaacatcgagagcgaaatgatcaaatttgccagttactatgctggaattgctgtcgcagtacttatcacaggatatattcaaatatgcttttgggtcattgccgcagctcgtcagatacagaaaatgagaaaattttactttaggagaataatgagaatggaaatagggtggtttgactgcaattcagtgggggagctgaatacaagattctctgatgatattaataaaatcaatgatgccatagctgaccaaatggcccttttcattcagcgcatgacctcgaccatctgtggtttcctgttgggatttttcaggggttggaaactgaccttggttattatttctgtcagccctctcattgggattggagcagccaccattggtctgagtgtgtccaagtttacggactatgagctgaaggcctatgccaaagcaggggtggtggctgatgaagtcatttcatcaatgagaacagtggctgcttttggtggtgagaaaagagaggttgaaaggtatgagaaaaatcttgtgttcgcccagcgttggggaattagaaaaggaatagtgatgggattctttactggattcgtgtggtgtctcatctttttgtgttatgcactggccttctggtacggctccacacttgtcctggatgaaggagaatatacaccaggaacccttgtccagattttcctcagtgtcatagtaggagctttaaatcttggcaatgcctctccttgtttggaagcctttgcaactggacgtgcagcagccaccagcatttttgagacaatagacaggaaacccatcattgactgcatgtcagaagatggttacaagttggatcgaatcaagggtgaaattgaattccataatgtgaccttccattatccttccagaccagaggtgaagattctaaatgacctcaacatggccattaaaccaggggaaatgacagctctggtaggacccagtggagctggaaaaagtacagcactgcaactcattcagcgattctatgacccctgtgaaggaatggtgaccgtggatggccatgacattcgctctcttaacattcagtggcttagagatcagattgggatagtggagcaagagccagttctgttctctaccaccattgcagaaaatattcgctatggcagagaagatgcaacaatggaagacatagtccaagctgccaaggaggccaatgcctacaacttcatcatggacctgccacagcaatttgacacccttgttggagaaggaggaggccagatgagtggtggccagaaacaaagggtagctatcgccagagccctcatccgaaatcccaagattctgcttttggacatggccacctcagctctggacaatgagagtgaagccatggtgcaagaagtgctgagtaagattcagcatgggcacacaatcatttcagttgctcatcgcttgtctacggtcagagctgcagataccatcattggttttgaacatggcactgcagtggaaagagggacccatgaagaattactggaaaggaaaggtgtttacttcactctagtgactttgcaaagccagggaaatcaagctcttaatgaagaggacataaaggatgcaactgaagatgacatgcttgcgaggacctttagcagagggagctaccaggatagtttaagggcttccatccggcaacgctccaagtctcagctttcttacctggtgcacgaacctccattagctgttgtagatcataagtctacctatgaagaagatagaaaggacaaggacattcctgtgcaggaagaagttgaacctgccccagttaggaggattctgaaattcagtgctccagaatggccctacatgctggtagggtctgtgggtgcagctgtgaacgggacagtcacacccttgtatgcctttttattcagccagattcttgggactttttcaattcctgataaagaggaacaaaggtcacagatcaatggtgtgtgcctactttttgtagcaatgggctgtgtatctcttttcacccaatttctacagggatatgcctttgctaaatctggggagctcctaacaaaaaggctacgtaaatttggtttcagggcaatgctggggcaagatattgcctggtttgatgacctcagaaatagccctggagcattgacaacaagacttgctacagatgcttcccaagttcaaggggctgccggctctcagatcgggatgatagtcaattccttcactaacgtcactgtggccatgatcattgccttctcctttagctggaagctgagcctggtcatcttgtgcttcttccccttcttggctttatcaggagccacacagaccaggatgttgacaggatttgcctctcgagataagcaggccctggagatggtgggacagattacaaatgaagccctcagtaacatccgcactgttgctggaattggaaaggagaggcggttcattgaagcacttgagactgagctggagaagcccttcaagacagccattcagaaagccaatatttacggattctgctttgcctttgcccagtgcatcatgtttattgcgaattctgcttcctacagatatggaggttacttaatctccaatgaggggctccatttcagctatgtgttcagggtgatctctgcagttgtactgagtgcaacagctcttggaagagccttctcttacaccccaagttatgcaaaagctaaaatatcagctgcacgcttttttcaactgctggaccgacaacccccaatcagtgtatacaatactgcaggtgaaaaatgggacaacttccaggggaagattgattttgttgattgtaaatttacatatccttctcgacctgactcgcaagttctgaatggtctctcagtgtcgattagtccagggcagacactggcgtttgttgggagcagtggatgtggcaaaagcactagcattcagctgttggaacgtttctatgatcctgatcaagggaaggtgatgatagatggtcatgacagcaaaaaagtaaatgtccagttcctccgctcaaacattggaattgtttcccaggaaccagtgttgtttgcctgtagcataatggacaatatcaagtatggagacaacaccaaagaaattcccatggaaagagtcatagcagctgcaaaacaggctcagctgcatgattttgtcatgtcactcccagagaaatatgaaactaacgttgggtcccaggggtctcaactctctagaggggagaaacaacgcattgctattgctcgggccattgtacgagatcctaaaatcttgctactagatgaagccacttctgccttagacacagaaagtgaaaagacggtgcaggttgctctagacaaagccagagagggtcggacctgcattgtcattgcccatcgcttgtccaccatccagaacgcggatatcattgctgtcatggcacagggggtggtgattgaaaaggggacccatgaagaactgatggcccaaaaaggagcctactacaaactagtcaccactggatcccccatcagttga

SEQ ID NO: 3 - DNA, 4650 bp, Homo sapiens
agtccaggaatcatgctggagaagttctgcaactctactttttggaattcctcattcctggacagtccggaggcagacctgccactttgttttgagcaaactgttctggtgtggattcccttgggcttcctatggctcctggccccctggcagcttctccacgtgtataaatccaggaccaagagatcctctaccaccaaactctatcttgctaagcaggtattcgttggttttcttcttattctagcagccatagagctggcccttgtactcacagaagactctggacaagccacagtccctgctgttcgatataccaatccaagcctctacctaggcacatggctcctggttttgctgatccaatacagcagacaatggtgtgtacagaaaaactcctggttcctgtccctattctggattctctcgatactctgtggcactttccaatttcagactctgatccggacactcttacagggtgacaattctaatctagcctactcctgcctgttcttcatctcctacggattccagatcctgatcctgatcttttcagcattttcagaaaataatgagtcatcaaataatccatcatccatagcttcattcctgagtagcattacctacagctggtatgacagcatcattctgaaaggctacaagcgtcctctgacactcgaggatgtctgggaagttgatgaagagatgaaaaccaagacattagtgagcaagtttgaaacgcacatgaagagagagctgcagaaagccaggcgggcactccagagacggcaggagaagagctcccagcagaactctggagccaggctgcctggcttgaacaagaatcagagtcaaagccaagatgcccttgtcctggaagatgttgaaaagaaaaaaaagaagtctgggaccaaaaaagatgttccaaaatcctggttgatgaaggctctgttcaaaactttctacatggtgctcctgaaatcattcctactgaagctagtgaatgacatcttcacgtttgtgagtcctcagctgctgaaattgctgatctcctttgcaagtgaccgtgacacatatttgtggattggatatctctgtgcaatcctcttattcactgcggctctcattcagtctttctgccttcagtgttatttccaactgtgcttcaagctgggtgtaaaagtacggacagctatcatggcttctgtatataagaaggcattgaccctatccaacttggccaggaaggagtacaccgttggagaaacagtgaacctgatgtctgtggatgcccagaagctcatggatgtgaccaacttcatgcacatgctgtggtcaagtgttctacagattgtcttatctatcttcttcctatggagagagttgggaccctcagtcttagcaggtgttggggtgatggtgcttgtaatcccaattaatgcgatactgtccaccaagagtaagaccattcaggtcaaaaatatgaagaataaagacaaacgtttaaagatcatgaatgagattcttagtggaatcaagatcctgaaatattttgcctgggaaccttcattcagagaccaagtacaaaacctccggaagaaagagctcaagaacctgctggcctttagtcaactacagtgtgtagtaatattcgtcttccagttaactccagtcctggtatctgtggtcacattttctgtttatgtcctggtggatagcaacaatattttggatgcacaaaaggccttcacctccattaccctcttcaatatcctgcgctttcccctgagcatgcttcccatgatgatctcctccatgctccaggccagtgtttccacagagcggctagagaagtacttgggaggggatgacttggacacatctgccattcgacatgactgcaattttgacaaagccatgcagttttctgaggcctcctttacctgggaacatgattcggaagccacagtccgagatgtgaacctggacattatggcaggccaacttgtggctgtgataggccctgtcggctctgggaaatcctccttgatatcagccatgctgggagaaatggaaaatgtccacgggcacatcaccatcaagggcaccactgcctatgtcccacagcagtcctggattcagaatggcaccataaaggacaacatcctttttggaacagagtttaatgaaaagaggtaccagcaagtactggaggcctgtgctctcctcccagacttggaaatgctgcctggaggagatttggctgagattggagagaagggtataaatcttagtgggggtcagaagcagcggatcagcctggccagagctacctaccaaaatttagacatctatcttctagatgaccccctgtctgcagtggatgctcatgtaggaaaacatatttttaataaggtcttgggccccaatggcctgttgaaaggcaagactcgactcttggttacacatagcatgcactttcttcctcaagtggatgagattgtagttctggggaatggaacaattgtagagaaaggatcctacagtgctctcctggccaaaaaaggagagtttgctaagaatctgaagacatttctaagacatacaggccctgaagaggaagccacagtccatgatggcagtgaagaagaagacgatgactatgggctgatatccagtgtggaagagatccccgaagatgcagcctccataaccatgagaagagagaacagctttcgtcgaacacttagccgcagttctaggtccaatggcaggcatctgaagtccctgagaaactccttgaaaactcggaatgtgaatagcctgaaggaagacgaagaactagtgaaaggacaaaaactaattaagaaggaattcatagaaactggaaaggtgaagttctccatctacctggagtacctacaagcaataggattgttttcgatattcttcatcatccttgcgtttgtgatgaattctgtggcttttattggatccaacctctggctcagtgcttggaccagtgactctaaaatcttcaatagcaccgactatccagcatctcagagggacatgagagttggagtctacggagctctgggattagcccaaggtatatttgtgttcatagcacatttctggagtgcctttggtttcgtccatgcatcaaatatcttgcacaagcaactgctgaacaatatccttcgagcacctatgagattttttgacacaacacccacaggccggattgtgaacaggtttgccggcgatatttccacagtggatgacaccctgcctcagtccttgcgcagctggattacatgcttcctggggataatcagcacccttgtcatgatctgcatggccactcctgtcttcaccatcatcgtcattcctcttggcattatttatgtatctgttcagatgttttatgtgtctacctcccgccagctgaggcgtctggactctgtcaccaggtccccaatctactctcacttcagcgagaccgtatcaggtttgccagttatccgtgcctttgagcaccagcagcgatttctgaaacacaatgaggtgaggattgacaccaaccagaaatgtgtcttttcctggatcacctccaacaggtggcttgcaattcgcctggagctggttgggaacctgactgtcttcttttcagccttgatgatggttatttatagagataccctaagtggggacactgttggctttgttctgtccaatgcactcaatatcacacaaaccctgaactggctggtgaggatgacatcagaaatagagaccaacattgtggctgttgagcgaataactgagtacacaaaagtggaaaatgaggcaccctgggtgactgataagaggcctccgccagattggcccagcaaaggcaagatccagtttaacaactaccaagtgcggtaccgacctgagctggatctggtcctcagagggatcacttgtgacattggtagcatggagaagattggtgtggtgggcaggacaggagctggaaagtcatccctcacaaactgcctcttcagaatcttagaggctgccggtggtcagattatcattgatggagtagatattgcttccattgggctccacgacctccgagagaagctgaccatcatcccccaggaccccatcctgttctctggaagcctgaggatgaatctcgaccctttcaacaactactcagatgaggagatttggaaggccttggagctggctcacctcaagtcttttgtggccagcctgcaacttgggttatcccacgaagtgacagaggctggtggcaacctgagcataggccagaggcagctgctgtgcctgggcagggctctgcttcggaaatccaagatcctggtcctggatgaggccactgctgcggtggatctagagacagacaacctcattcagacgaccatccaaaacgagttcgcccactgcacagtgatcaccatcgcccacaggctgcacaccatcatggacagtgacaaggtaatggtcctagacaacgggaagattatagagtgcggcagccctgaagaactgctacaaatccctggacccttttactttatggctaaggaagctggcattgagaatgtgaacagcacaaaattctag

SEQ ID NO: 4 - DNA, 6433 bp, Artificial Sequence
  Plasmid
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatcccaggaggatggaggcccacaacgcgtctgccccattcaacttcaccctgccacccaactttggcaagcgccccacagacctggcactgagcgtcatcctggtgttcatgttgttcttcatcatgctctcgctgggctgcaccatggagttcagcaagatcaaggctcacttatggaagcctaaagggctggccatcgccctggtggcacagtatggcatcatgcccctcacggcctttgtgctgggcaaggtcttccggctgaagaacattgaggcactggccatcttggtctgtggctgctcacctggagggaacctgtccaatgtcttcagtctggccatgaagggggacatgaacctcagcattgtgatgaccacctgctccaccttctgtgcccttggcatgatgcctctcctcctgtacatctactccagggggatctatgatggggacctgaaggacaaggtgccctataaaggcatcgtgatatcactggtcctggttctcattccttgcaccatagggatcgtcctcaaatctaaacggccacaatacatgcgctatgtcatcaagggagggatgatcatcattctcttgtgcagtgtggccgtcacagttctctctgccatcaatgtggggaagagcatcatgtttgccatgacaccactcttgattgccacctcctccctgatgccttctattggctttctgctgggttatgttctctctgctctcttctgcctcaatggacggtgcagacgcactgtcagcatggagactggatgccaaaatgtccaactctgttccaccatcctcaatgtggcctttccacctgaagtcattggaccacttttcttctttcccctcctctacatgattttccagcttggagaagggcttctcctcattgccatattttggtgctatgagaaattcaagactcccaaggataaaacaaaaatgatctacacagctgccacaactgaagaaacaattccaggagctctgggaaatggcacctacaaaggggaggactgctccccttgcacagcctagcccttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 5 - DNA, 9043 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagctatcacaagtttgtacaaaaaagcaggcttaggaatgtctgactcagtaattcttcgaagtataaagaaatttggagaggagaatgatggttttgagtcagataaatcatataataatgataagaaatcaaggttacaagatgagaagaaaggtgatggcgttagagttggcttctttcaattgtttcggttttcttcatcaactgacatttggctgatgtttgtgggaagtttgtgtgcatttctccatggaatagcccagccaggcgtgctactcatttttggcacaatgacagatgtttttattgactacgacgttgagttacaagaactccagattccaggaaaagcatgtgtgaataacaccattgtatggactaacagttccctcaaccagaacatgacaaatggaacacgttgtgggttgctgaacatcgagagcgaaatgatcaaatttgccagttactatgctggaattgctgtcgcagtacttatcacaggatatattcaaatatgcttttgggtcattgccgcagctcgtcagatacagaaaatgagaaaattttactttaggagaataatgagaatggaaatagggtggtttgactgcaattcagtgggggagctgaatacaagattctctgatgatattaataaaatcaatgatgccatagctgaccaaatggcccttttcattcagcgcatgacctcgaccatctgtggtttcctgttgggatttttcaggggttggaaactgaccttggttattatttctgtcagccctctcattgggattggagcagccaccattggtctgagtgtgtccaagtttacggactatgagctgaaggcctatgccaaagcaggggtggtggctgatgaagtcatttcatcaatgagaacagtggctgcttttggtggtgagaaaagagaggttgaaaggtatgagaaaaatcttgtgttcgcccagcgttggggaattagaaaaggaatagtgatgggattctttactggattcgtgtggtgtctcatctttttgtgttatgcactggccttctggtacggctccacacttgtcctggatgaaggagaatatacaccaggaacccttgtccagattttcctcagtgtcatagtaggagctttaaatcttggcaatgcctctccttgtttggaagcctttgcaactggacgtgcagcagccaccagcatttttgagacaatagacaggaaacccatcattgactgcatgtcagaagatggttacaagttggatcgaatcaagggtgaaattgaattccataatgtgaccttccattatccttccagaccagaggtgaagattctaaatgacctcaacatggccattaaaccaggggaaatgacagctctggtaggacccagtggagctggaaaaagtacagcactgcaactcattcagcgattctatgacccctgtgaaggaatggtgaccgtggatggccatgacattcgctctcttaacattcagtggcttagagatcagattgggatagtggagcaagagccagttctgttctctaccaccattgcagaaaatattcgctatggcagagaagatgcaacaatggaagacatagtccaagctgccaaggaggccaatgcctacaacttcatcatggacctgccacagcaatttgacacccttgttggagaaggaggaggccagatgagtggtggccagaaacaaagggtagctatcgccagagccctcatccgaaatcccaagattctgcttttggacatggccacctcagctctggacaatgagagtgaagccatggtgcaagaagtgctgagtaagattcagcatgggcacacaatcatttcagttgctcatcgcttgtctacggtcagagctgcagataccatcattggttttgaacatggcactgcagtggaaagagggacccatgaagaattactggaaaggaaaggtgtttacttcactctagtgactttgcaaagccagggaaatcaagctcttaatgaagaggacataaaggatgcaactgaagatgacatgcttgcgaggacctttagcagagggagctaccaggatagtttaagggcttccatccggcaacgctccaagtctcagctttcttacctggtgcacgaacctccattagctgttgtagatcataagtctacctatgaagaagatagaaaggacaaggacattcctgtgcaggaagaagttgaacctgccccagttaggaggattctgaaattcagtgctccagaatggccctacatgctggtagggtctgtgggtgcagctgtgaacgggacagtcacacccttgtatgcctttttattcagccagattcttgggactttttcaattcctgataaagaggaacaaaggtcacagatcaatggtgtgtgcctactttttgtagcaatgggctgtgtatctcttttcacccaatttctacagggatatgcctttgctaaatctggggagctcctaacaaaaaggctacgtaaatttggtttcagggcaatgctggggcaagatattgcctggtttgatgacctcagaaatagccctggagcattgacaacaagacttgctacagatgcttcccaagttcaaggggctgccggctctcagatcgggatgatagtcaattccttcactaacgtcactgtggccatgatcattgccttctcctttagctggaagctgagcctggtcatcttgtgcttcttccccttcttggctttatcaggagccacacagaccaggatgttgacaggatttgcctctcgagataagcaggccctggagatggtgggacagattacaaatgaagccctcagtaacatccgcactgttgctggaattggaaaggagaggcggttcattgaagcacttgagactgagctggagaagcccttcaagacagccattcagaaagccaatatttacggattctgctttgcctttgcccagtgcatcatgtttattgcgaattctgcttcctacagatatggaggttacttaatctccaatgaggggctccatttcagctatgtgttcagggtgatctctgcagttgtactgagtgcaacagctcttggaagagccttctcttacaccccaagttatgcaaaagctaaaatatcagctgcacgcttttttcaactgctggaccgacaacccccaatcagtgtatacaatactgcaggtgaaaaatgggacaacttccaggggaagattgattttgttgattgtaaatttacatatccttctcgacctgactcgcaagttctgaatggtctctcagtgtcgattagtccagggcagacactggcgtttgttgggagcagtggatgtggcaaaagcactagcattcagctgttggaacgtttctatgatcctgatcaagggaaggtgatgatagatggtcatgacagcaaaaaagtaaatgtccagttcctccgctcaaacattggaattgtttcccaggaaccagtgttgtttgcctgtagcataatggacaatatcaagtatggagacaacaccaaagaaattcccatggaaagagtcatagcagctgcaaaacaggctcagctgcatgattttgtcatgtcactcccagagaaatatgaaactaacgttgggtcccaggggtctcaactctctagaggggagaaacaacgcattgctattgctcgggccattgtacgagatcctaaaatcttgctactagatgaagccacttctgccttagacacagaaagtgaaaagacggtgcaggttgctctagacaaagccagagagggtcggacctgcattgtcattgcccatcgcttgtccaccatccagaacgcggatatcattgctgtcatggcacagggggtggtgattgaaaaggggacccatgaagaactgatggcccaaaaaggagcctactacaaactagtcaccactggatcccccatcagttgagacccagctttcttgtacaaagtggtgattggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 6 - DNA, 9658 bp, Artificial Sequence
  Plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcagtccaggaatcatgctggagaagttctgcaactctactttttggaattcctcattcctggacagtccggaggcagacctgccactttgttttgagcaaactgttctggtgtggattcccttgggcttcctatggctcctggccccctggcagcttctccacgtgtataaatccaggaccaagagatcctctaccaccaaactctatcttgctaagcaggtattcgttggttttcttcttattctagcagccatagagctggcccttgtactcacagaagactctggacaagccacagtccctgctgttcgatataccaatccaagcctctacctaggcacatggctcctggttttgctgatccaatacagcagacaatggtgtgtacagaaaaactcctggttcctgtccctattctggattctctcgatactctgtggcactttccaatttcagactctgatccggacactcttacagggtgacaattctaatctagcctactcctgcctgttcttcatctcctacggattccagatcctgatcctgatcttttcagcattttcagaaaataatgagtcatcaaataatccatcatccatagcttcattcctgagtagcattacctacagctggtatgacagcatcattctgaaaggctacaagcgtcctctgacactcgaggatgtctgggaagttgatgaagagatgaaaaccaagacattagtgagcaagtttgaaacgcacatgaagagagagctgcagaaagccaggcgggcactccagagacggcaggagaagagctcccagcagaactctggagccaggctgcctggcttgaacaagaatcagagtcaaagccaagatgcccttgtcctggaagatgttgaaaagaaaaaaaagaagtctgggaccaaaaaagatgttccaaaatcctggttgatgaaggctctgttcaaaactttctacatggtgctcctgaaatcattcctactgaagctagtgaatgacatcttcacgtttgtgagtcctcagctgctgaaattgctgatctcctttgcaagtgaccgtgacacatatttgtggattggatatctctgtgcaatcctcttattcactgcggctctcattcagtctttctgccttcagtgttatttccaactgtgcttcaagctgggtgtaaaagtacggacagctatcatggcttctgtatataagaaggcattgaccctatccaacttggccaggaaggagtacaccgttggagaaacagtgaacctgatgtctgtggatgcccagaagctcatggatgtgaccaacttcatgcacatgctgtggtcaagtgttctacagattgtcttatctatcttcttcctatggagagagttgggaccctcagtcttagcaggtgttggggtgatggtgcttgtaatcccaattaatgcgatactgtccaccaagagtaagaccattcaggtcaaaaatatgaagaataaagacaaacgtttaaagatcatgaatgagattcttagtggaatcaagatcctgaaatattttgcctgggaaccttcattcagagaccaagtacaaaacctccggaagaaagagctcaagaacctgctggcctttagtcaactacagtgtgtagtaatattcgtcttccagttaactccagtcctggtatctgtggtcacattttctgtttatgtcctggtggatagcaacaatattttggatgcacaaaaggccttcacctccattaccctcttcaatatcctgcgctttcccctgagcatgcttcccatgatgatctcctccatgctccaggccagtgtttccacagagcggctagagaagtacttgggaggggatgacttggacacatctgccattcgacatgactgcaattttgacaaagccatgcagttttctgaggcctcctttacctgggaacatgattcggaagccacagtccgagatgtgaacctggacattatggcaggccaacttgtggctgtgataggccctgtcggctctgggaaatcctccttgatatcagccatgctgggagaaatggaaaatgtccacgggcacatcaccatcaagggcaccactgcctatgtcccacagcagtcctggattcagaatggcaccataaaggacaacatcctttttggaacagagtttaatgaaaagaggtaccagcaagtactggaggcctgtgctctcctcccagacttggaaatgctgcctggaggagatttggctgagattggagagaagggtataaatcttagtgggggtcagaagcagcggatcagcctggccagagctacctaccaaaatttagacatctatcttctagatgaccccctgtctgcagtggatgctcatgtaggaaaacatatttttaataaggtcttgggccccaatggcctgttgaaaggcaagactcgactcttggttacacatagcatgcactttcttcctcaagtggatgagattgtagttctggggaatggaacaattgtagagaaaggatcctacagtgctctcctggccaaaaaaggagagtttgctaagaatctgaagacatttctaagacatacaggccctgaagaggaagccacagtccatgatggcagtgaagaagaagacgatgactatgggctgatatccagtgtggaagagatccccgaagatgcagcctccataaccatgagaagagagaacagctttcgtcgaacacttagccgcagttctaggtccaatggcaggcatctgaagtccctgagaaactccttgaaaactcggaatgtgaatagcctgaaggaagacgaagaactagtgaaaggacaaaaactaattaagaaggaattcatagaaactggaaaggtgaagttctccatctacctggagtacctacaagcaataggattgttttcgatattcttcatcatccttgcgtttgtgatgaattctgtggcttttattggatccaacctctggctcagtgcttggaccagtgactctaaaatcttcaatagcaccgactatccagcatctcagagggacatgagagttggagtctacggagctctgggattagcccaaggtatatttgtgttcatagcacatttctggagtgcctttggtttcgtccatgcatcaaatatcttgcacaagcaactgctgaacaatatccttcgagcacctatgagattttttgacacaacacccacaggccggattgtgaacaggtttgccggcgatatttccacagtggatgacaccctgcctcagtccttgcgcagctggattacatgcttcctggggataatcagcacccttgtcatgatctgcatggccactcctgtcttcaccatcatcgtcattcctcttggcattatttatgtatctgttcagatgttttatgtgtctacctcccgccagctgaggcgtctggactctgtcaccaggtccccaatctactctcacttcagcgagaccgtatcaggtttgccagttatccgtgcctttgagcaccagcagcgatttctgaaacacaatgaggtgaggattgacaccaaccagaaatgtgtcttttcctggatcacctccaacaggtggcttgcaattcgcctggagctggttgggaacctgactgtcttcttttcagccttgatgatggttatttatagagataccctaagtggggacactgttggctttgttctgtccaatgcactcaatatcacacaaaccctgaactggctggtgaggatgacatcagaaatagagaccaacattgtggctgttgagcgaataactgagtacacaaaagtggaaaatgaggcaccctgggtgactgataagaggcctccgccagattggcccagcaaaggcaagatccagtttaacaactaccaagtgcggtaccgacctgagctggatctggtcctcagagggatcacttgtgacattggtagcatggagaagattggtgtggtgggcaggacaggagctggaaagtcatccctcacaaactgcctcttcagaatcttagaggctgccggtggtcagattatcattgatggagtagatattgcttccattgggctccacgacctccgagagaagctgaccatcatcccccaggaccccatcctgttctctggaagcctgaggatgaatctcgaccctttcaacaactactcagatgaggagatttggaaggccttggagctggctcacctcaagtcttttgtggccagcctgcaacttgggttatcccacgaagtgacagaggctggtggcaacctgagcataggccagaggcagctgctgtgcctgggcagggctctgcttcggaaatccaagatcctggtcctggatgaggccactgctgcggtggatctagagacagacaacctcattcagacgaccatccaaaacgagttcgcccactgcacagtgatcaccatcgcccacaggctgcacaccatcatggacagtgacaaggtaatggtcctagacaacgggaagattatagagtgcggcagccctgaagaactgctacaaatccctggacccttttactttatggctaaggaagctggcattgagaatgtgaacagcacaaaattctagcttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

(19) United States
(12) Patent Application Publication
(10) Pub. No.: US 2007/0287167 A1
(43) Pub. Date: Dec. 13, 2007

(54) Dual-Transfected Cells Lines As In Vitro Screening Tools For Pharmaceutical Compound Profiling: A Model For The Heptobiliary Elimination

(76) Inventors: Stefan Theis, Frankfurt (DE); Tanja Eisenblaetter, Frankfurt (DE)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(21) Appl. No.: 10/594,419

(22) PCT Filed: Apr. 8, 2005
(86) PCT No.: PCT/EP05/03715

§ 371 Date: May 22, 2007

(30) Foreign Application Priority Data
Apr. 16, 2004 (DE) 102004018608.1

(51) Int. Cl. C12N 5/06; C12M 1/22; C12N 15/87; C12Q 1/02; C12N 5/02

(52) U.S. Cl. 435/29; 435/305.1; 435/325; 435/350; 435/378; 435/395; 435/465

(57) ABSTRACT

The invention is referring to several dual-transfected cell lines expressing human NTCP (Na/taurocholate Cotransporting Protein; SLC10A1) together with human BSEP (Bile Salt Export Pump; ABCB11) or human MRP2 (Multidrug Resistance Protein; ABCC2) suitable as an in vitro tool for pharmaceutical compound profiling particularly as a model for hepatobiliary elimination.

A cell line transfected with both human NTCP and human MRP2 suitable as an in vitro tool for pharmaceutical compound profiling: A model for hepatobiliary elimination

The invention is referring to several dual-transfected cell lines expressing human NTCP (Na/taurocholate Cotransporting Protein; SLC10A1) together with human BSEP (Bile Salt Export Pump; ABCB11) or human MRP2 (Multidrug Resistance Protein; ABCC2) suitable as an in vitro tool for pharmaceutical compound profiling particularly as a model for hepatobiliary elimination.

Transcellular transport processes across biological barriers are playing an essential role in the absorption, distribution and elimination of drugs and xenobiotics. In intestinal epithelial cells transport proteins are involved in the absorption or exclusion of drugs into/from the systemic circulation. Transport proteins expressed in the cells of the blood-brain-barrier are involved in the excision of toxic (and also therapeutic) compounds from the brain. In kidney proximal tubule epithelia cells and in hepatocytes transport proteins are involved in the elimination of various endogenous and exogenous substances. In the hepatocyte this elimination process is mediated by (i) uptake from blood into the cell via the basolateral membrane and (ii) elimination into the bile via the apical membrane. Paracellular diffusion in all biological barriers is anticipated by the formation of tight junctions (molecular structures providing a very tight contact between adjacent cells) separating the apical from the basolateral membrane compartment. Cell lines that have maintained the features of a biological barrier (development of basolateral and apical membrane compartments separated by tight junctions) are also called polarized cell lines. Usually these cell lines grow in a single cell layer designated as monolayer.

The basolateral (also called sinusoidal) uptake of compounds into the human hepatocyte is mediated by a variety of different transport proteins: Bulky organic anions are taken up by OATP8 (SLC21A8), OATP2 (SLC21A6, also known as OATP-C or LST1) and OATP-B (SLC21A9) whereas bile acids and possibly other compounds, similar to bile acids are accepted by the Sodium Taurocholate Co-transporting Polypeptide NTCP (SLC10A1). OAT2 (SLC22A7) imports small organic anions into the hepatocyte and OCT1 (SLC22A1) has been reported to facilitate the cellular uptake of small organic cations. At the apical (also called canalicular) membrane, the elimination of organic anions and bile salts into bile is mediated by the Multidrug-resistance Related Protein MRP2 (ABCC2) and the Bile Salt Export Pump BSEP (ABCB11), both members of the ABC transporter family (ATP-dependent export pumps). Other ABC Transporters also known to be expressed at the canalicular membrane of the human hepatocyte are the Breast Cancer Resistance Protein BCRP (ABCG2, also known as MXR) and the Multidrug-resistance protein MDR1 (ABCB1, also known as P-glycoprotein). These two proteins are thought to participate in the canalicular secretion process of amphiphilic and cationic compounds.

The multidrug resistance protein (MDR) family mediates the ATP-dependent unidirectional transport of conjugates of lipophilic substances with glutathione, glucuronate, or sulfate (Review: König J. et al., Biochimica et Biophysica Acta 1461 (1999) 377-394).

The bile salt pool undergoes an enterohepatic circulation that is regulated by distinct bile salt transport proteins, including the canalicular bile salt export pump BSEP, the ileal NA⁺-depentent bile salt transporter ISBT, and the hepatic sinusoidal Na⁺-taurocholate cotransporting polypeptide NTCP. Other bile salt transporters include the organic anion transporting polypeptides OATPs and the multidrug resistance-associatd proteins 2 and 3 MRP2,2 (Review: Trauner et al., Physiol. Rev. 83: 633-671, 2003).

To date the only way to investigate hepatobiliary elimination of drugs is the bile fistula study done in rats, a complex and time-consuming in vivo assay. In addition the bile fistula study doesn't give any information about the molecular basis of the elimination process. Here we describe a cell-based assay system in which a liver uptake transporter (in this case human NTCP) is constitutively expressed together with a liver export pump (usually an ABC transporter, in this case human MRP2 or human BSEP) in the polarized canine kidney cell line MDCKII (for reference see Louvard, 1980; Fuller et al., 1984). The resulting cell lines therefore express two transgenes in one cell line (i) human NTCP together with human BSEP (MDCKII-hNTCP/hBSEP) and (ii) human NTCP together with human MRP2 (MDCKII-hNTCP-hMRP2). The cells are cultivated in 6-well filter inserts on a porous filter membrane thereby separating a basolateral from an apical compartment (FIG. 1). Putative substrates (e.g. pharmaceutical compounds) are added either to the apical or to the basolateral compartment for transcellular (vectorial) transport measurements in both directions. Samples are taken from each compartment after the indicated incubation periods. Substrates of a given transporter combination (in this case human NTCP and human BSEP or human NTCP and human MRP2) display a significant net transport from the basolateral to the apical compartment when compared to un-transfected or mono-transfected cells (FIG. 4+5).

With the aid of the described dual-transfected cell lines one will be able to predict whether the transporter combinations human NTCP/human BSEP or human NTCP/human MRP2 participates in the hepatobiliary elimination process of a pharmaceutical compound or not.

During pharmaceutical drug development compounds frequently fail to proceed into clinical development due to their weak pharmacokinetic profile. One reason for this is their rapid elimination via the liver into the bile. Examples for this phenomenon are the chlorogenic acid derivatives, potent and specific inhibitors of the glucose-6-phosphate translocase (a potential novel pharmaceutical target in patients with type II diabetes). Pharmacodynamic studies showed that the blood glucose lowering effect of several chlorogenic acid derivatives lasts only a short time after bolus i.v. injection in Wistar rats. Bile fistula studies indicated that this weak pharmacodynamic effect is the result of unsuitable pharmacokinetics due to a rapid hepatobiliary elimination process. With the aid of dual-transfected transporter cell lines one will be able to identify the transporters involved in the elimination process. In a next step the compounds can than be chemically modified in a way that alleviates their affinity to the eliminating transporter to improve the compounds pharmacology.

Another application area would be the examination of potential drug-drug interactions at the level of transporters in the liver. For example competition of two drugs for one transporter in the liver may alter the pharmacokinetic profile of both compounds significantly. In addition, inhibition or activation of a transporter by one drug may also change the pharmacological behaviour of the transporters substrates.

The invention pertains to a mammalian cell having a first and a second side which both sides form part of the outer surface of such cell and which both sides are different from the areas of contact of such cell and which first and second side are distinguished from each other by their localization at opposite ends of such cell wherein the first side carries a functional hNTCP protein and the second side carries a functional hBSEP protein.

The first side of such a mammalian cell is e.g. the basolateral side and the second side is the apical side or the first side is e.g. the apical side and the second side is e.g. the basolateral side.

The mammalian cell can be chosen from epithelial cells in particular of the kidney, of the bowels systems, of the liver or of the blood/brain barrier.

Such cells can be immortalized cells or recombinant cells. The cells can be taken from ordinary mammalian tissue or a primary cell culture. Such cells can be also the cells of a cell culture as e.g. LLC-PK1 cells or MDCKII cells in particular also when carrying recombinant vectors.

The LLC-PK1 cells may harbor for example a recombinant vector e.g. suitable for expressing the hNTCP protein (as exemplified by SEQ ID NO. 4) and a recombinant vector suitable for expressing the hBSEP protein (as exemplified by SEQ ID NO. 5). As alternative the hNTCP and the hBSEP proteins may be expressed simultaneously from the same vector construct. The MDCKII cell may harbor for example a recombinant vector e.g. suitable for expressing the hNTCP protein (as exemplified by SEQ ID NO. 4 ) and a recombinant vector suitable for expressing the hBSEP protein (as exemplified by SEQ ID NO. 5 ). Such a mammalian cell of a MDCKII type carrying two separate vectors with ability of expressing hNTCP protein and hBSEP protein has been deposited with the DSMZ-Deutsche Sammlung von Mikroorganismen und Zelikulturen GmbH, Mascheroder Weg 1, D-38124 Braunschweig. The identification number is DSM ACC2643.

The invention pertains further to the manufacturing of a mammalian cell carrying a functional human NTCP protein and a functional human BSEP protein wherein
a] a mammalian cell is provided;

b] a vector is provided encompassing the coding sequence of hNTCP (e.g. vector as drafted according or equal to SEQ ID NO. 4 );

c] a vector is provided encompassing the coding sequence of hBSEP (e.g. vector as drafted according or equal to SEQ ID NO. 5);

d] the mammalian cell from a] is transformed by a vector from b] and by a vector from c] either simultaneously or consecutively;

e] a double transfectant cell from d] is identified and propagated.

The mammalian cells that shall be provided for such a manufacturing may be epithelial cells in particular of the kidney, of the bowels system, of the liver or of the blood/brain barrier. Such cells can be also immortalized cells or recombinant cells. The cells can be taken from ordinary mammalian tissue or a primary cell culture. Such double transfectant cells according to e] resulting from the manufacturing as mentioned above may be e.g. LLC-PK1 cells or MDCKII cells in particular when carrying recombinant vectors. Such LLC-PK1 cells may harbor for example a recombinant vector e.g. suitable for expressing the hNTCP protein (as exemplified by SEQ ID NO. 4 ) and a recombinant vector suitable for expressing the hBSEP protein (as exemplified by SEQ ID NO. 5). The hNTCP and the hBSEP proteins may be expressed also simultaneously from the same vector construct. Such MDCKII cells may harbor for example a recombinant vector e.g. suitable for expressing the hNTCP protein (as exemplified by SEQ ID NO. 4 ) and a recombinant vector suitable for expressing the hBSEP (as exemplified by SEQ ID NO. 5). Such a mammalian cell of a MDCKII type carrying two separate vectors with ability of expressing hNTCP protein and hBSEP protein has been deposited with the DSMZ-Deutsche Sammlung von Mikroorganismen and Zellkulturen GmbH, Mascheroder Weg 1 b, D-38124 Braunschweig. The identification number is DSM ACC2643.

The invention pertains further to a monolayer of cells comprising at least two cells of mammalian cells having a first and a second side which both sides form part of the outer surface of such cell and which both sides are different from the areas of contact of such cell and which first and second side are distinguished from each other by their localization at opposite ends of such cell wherein the first side carries a functional hNTCP protein and the second side carries a functional hBSEP protein. Such a monolayer could occupy a part or the whole of a solid surface. The invention pertains also to such a solid surface carrying a monolayer of such cells.

The solid surface could be formed by a plastic. The solid surface could also be part of a petri dish or a filter-insert.

The invention pertains further to a petri dish carrying a monolayer of cells as described before and as well as to a filter-insert carrying a monolayer of cells as described before. The membrane support of such a filter-insert could be made of polycarbonate and/or polyester. The pore size of the membrane support could be of 0.4 μm+/−0.2 μm. An according example of a filter-insert has been depicted in FIG. 1. There are several manufacturers of filter-inserts that could be used for placing the said monolayer of cells on it. Such manufacturers are for example “Corning Inc., Corning, N.Y.” or “Millipore”.

The mammalian cell having a first and a second side which both sides form part of the outer surface of such cell and which both sides are different from the areas of contact of such cell and which first and second side are distinguished from each other by their localization at opposite ends of such cell wherein the first side carries a functional hNTCP protein and the second side carries a functional hBSEP protein could be used e.g. for determining pharmacological profiles with respect to hepatobiliary elimination and/or renal excretion and/or brain resorption and/or intestinal resorption. For such use the mammalian cells could form part of a monolayer on a solid surface and/or a petri dish and/or on a filter insert.

The invention pertains further to a mammalian cell having a first and a second side which both sides form part of the outer surface of such cell and which both sides are different from the areas of contact of such cell and which first and second side are distinguished from each other by their localization at opposite ends of such cell wherein the first side carries a functional hNTCP protein and the second side carries a functional hMRP2 protein.

The first side of such a mammalian cell is e.g. the basolateral side and the second side is the apical side or the first side is e.g. the apical side and the second side is e.g. the basolateral side.

The mammalian cell can be chosen from epithelial cells in particular of the kidney, of the bowels system, of the liver or of the blood/brain barrier.

Such cells can be immortalized cells or recombinant cells. The cells can be taken from ordinary mammalian tissue or a primary cell culture. Such cells can be also the cells of a cell culture as e.g. LLC-PK1 cells or MDCKII cells particularly when carrying recombinant vectors.

The LLC-PK1 cells may harbor for example a recombinant vector e.g. suitable for expressing the hNTCP protein (as exemplified by SEQ ID NO. 4) and a recombinant vector suitable for expressing the hMRP2 protein (as exemplified by SEQ ID NO. 6). As alternative the hNTCP and the hMRP2 proteins may be expressed simultaneously from the same vector construct. The MDCKII cell shall harbor a recombinant vector e.g. suitable for expressing the hNTCP protein (as exemplified by SEQ ID NO. 4) and a recombinant vector suitable for expressing the hMRP2 protein (as exemplified by SEQ ID NO. 6). Such a mammalian cell of a MDCKII type carrying two separate vectors with ability of expressing hNTCP protein and hMRP2 protein has been deposited with the DSMZ-Deutsche Sammlung von Mikroorganismen und Zellkulturen GmbH, Mascheroder Weg 1 b, D-38124 Braunschweig. The identification number is DSM ACC2644.

The invention pertains further to the manufacturing of a mammalian cell carrying a functional human NTCP protein and a functional human MRP2 protein wherein

a] a mammalian cell is provided;

b] a vector is provided encompassing the coding sequence of hNTCP (e.g. a vector as drafted according or equal to SEQ ID NO. 4);

c] a vector is provided encompassing the coding sequence of hMRP2 (e.g. vector as drafted according or equal to SEQ ID NO. 6);

d] the mammalian cell from a] is transformed by a vector from b] and by a vector from c] either simultaneously or consecutively;

e] a double transfectant cell from d] is identified and propagated.

The mammalian cells that shall be provided for such a manufacturing may be epithelial cells in particular of the kidney, of the bowels system, of the liver or of the blood/brain barrier. Such cells can be also immortalized cells or recombinant cells. The cells can be taken from ordinary mammalian tissue or a primary cell culture.

Such double transfectant cells according to e] resulting from the manufacturing as mentioned above may be the cells of a cell culture as e.g. LLC-PK1 cells or MDCKII cells particularly when carrying recombinant vectors. Such LLC-PK1 cells may harbor for example a recombinant vector e.g. suitable for expressing the hNTCP protein (as exemplified by SEQ ID NO. 4) and a recombinant vector suitable for expressing the hMRP2 protein (as exemplified by SEQ ID NO. 6). The hNTCP and the hMRP2 proteins may be expressed also simultaneously from the same vector construct. Such MDCKII may harbor for example a recombinant vector e.g. suitable for expressing the hNTCP protein (as exemplified by SEQ ID NO. 4 ) and a recombinant vector suitable for expressing the hMRP2 (as exemplified by SEQ ID NO. 6). Such a mammalian cell of a MDCKII type carrying two separate vectors with ability of expressing hNTCP protein and hMRP2 protein has been deposited with the DSMZ—Deutsche Sammlung von Mikroorganismen und Zellkulturen GmbH, Mascheroder Weg 1 b, D-38124 Braunschweig. The identification number is DSM ACC2644.

The invention pertains further to a monolayer of cells comprising at least two cells of mammalian cells having a first and a second side which both sides form part of the outer surface of such cell and which both sides are different from the areas of contact of such cell and which first and second side are distinguished from each other by their localization at opposite ends of such cell wherein the first side carries a functional hNTCP protein and the second side carries a functional hMRP2 protein.

Such a monolayer could occupy a part or the whole of a solid surface. The invention pertains to such a solid surface carrying a monolayer of such cells.

The solid surface could be formed by a plastic. The solid surface could also be part of a petri dish or a filter-insert.

The invention pertains also to a petri dish carrying a monolayer of cells as described and further to a filter-insert carrying a monolayer of cells as described. The membrane support of such a filter-insert could be made of polycarbonate and/or polyester. The pore size of the membrane support could be of about 0.4 μm+/−0.2 μm. An according example of a filter-insert has been depicted in FIG. 1. There are several manufacturers of filter-inserts that could be used for placing the said monolayer of cells on it. Such manufacturers are for example “Corning Inc., Corning, N.Y.” or “Millipore”.

The mammalian cell having a first and a second side which both sides form part of the outer surface of such cell and which both sides are different from the areas of contact of such cell and which first and second side are distinguished from each other by their localization at opposite ends of such cell wherein the first side carries a functional hNTCP protein and the second side carries a functional hMRP2 protein could be used e.g. for determining pharmacological profiles with respect to hepatobiliary elimination and/or renal excretion and/or brain resorption and/or intestinal resorption. For such use the mammalian cells could form part of a monolayer on a solid surface and/or a petri dish and/or on a filter-insert.

A protein shall be regarded as functional in context of this invention when it is in a condition to perform an activity in a biological context in particular as part of a living cell. Such an activity is detectable e.g. by an assy. A transporter is functional for example when this transporter moves a compound in particular the transporter's biological substrate from outside a cell into the inner compartment of this cell or vice versa. A biological substrate of an ion transporter protein consists e.g. of a monovalent and/or a divalent ion or other ions. The substrate of a glucose transporter protein is e.g. glucose. The substrate of a multiple drug resistance protein is e.g. a drug alone or conjugated to gluthathion or gluconate.

The handling of proteins in context of this invention can be achieved by a person skilled in the art by applying the according protocols from “Current Protocols in Protein Science” published by John Wiley & Sons (edited by: John E. Coligan, Ben M. Dunn, Hidde L. Ploegh, David W. Speicher, Paul T. Wingfield; 0-471-11184-8-Looseleaf; 0-471-14098-8-CDROM).

The handling of techniques concerning Molecular Biology as e.g. cloning, transforming of cells, sequencing, modifying promoters, expressing proteins or others can be achieved a the person skilled in the art by applying the according protocols from “Current Protocols in Molecular Biology” published by John Wiley & Sons (edited by: Fred M. Ausubel, Roger Brent, Robert E. Kingston, David D. Moore, J. G. Seidman, John A. Smith, Kevin Struhl; 0-471-50338-X-Looseleaf; 0-471-306614CDROM).

The handling of biological cells can be achieved a the person skilled in the art by applying the according protocols from “Current Protocols in Cell Biology” published by John Wiley & Sons (edited by: Juan S. Bonifacino, Mary Dasso, Jennifer Lippincott-Schwartz, Joe B. Harford, Kenneth M. Yamada; 0-471-24108-3-Looseleaf; 0-471-24105-9-CDROM).

### EXAMPLES

### Cell Line Generation: MDCKII-hBSEP And MDCKII-hNTCP/hBSEP

MDCK II cells (Madin-Darby canine kidney cells, strain II) were cultured in Dulbecco's modified essential medium supplemented with 10% fetal calf serum at 37° C., 5% CO₂, and 95% humidity. The MDCKII-hNTCP/hBSEP cell line (DSM ACC2643) was generated as follows: MDCK II cells were first transfected with the mammalian expression plasmid pcDNA3.1zeo(+)-hBSEP using the FuGene6 transfection reagent as described by the manufacturer (Roche). Transfected cells were selected with 1000 μg/ml Zeocin. During selection the tissue culture medium was additionally supplemented with 100 units/ml penicillin G and 100 μg/ml streptomycin sulfate. The resulting cell clones were screened in the immunoblot for recombinant human BSEP expression followed by analysis of the proper subcellular localization of recombinant human BSEP using Confocal Laser Scanning Microscopy. The cell clone with the highest expression of human BSEP and concomitant localization of the recombinant BSEP at the apical membrane was chosen for FuGene6 transfection with the mammalian expression plasmid pcDNA3.1neo(+)-hNTCP. Transfected cells were selected with 800 μg/ml Geneticin and 1000 μg/ml Zeocin. During selection the tissue culture medium was additionally supplemented with 100 units/ml penicillin G and 100 μg/ml streptomycin sulfate. Double-transfected cell lines were again screened in the immunoblot for the expression of both, recombinant human BSEP and recombinant human NTCP, followed by analysis of the proper subcellular localization of both transgenes by Confocal Laser Scanning Microscopy. The cell clone with the highest expression and proper subcellular localization of both, human BSEP (apical membrane) and human NTCP (basolateral membrane) was chosen for further examinations.

### Cell Line Generation: MDCKII-hMRP2 And MDCKII-hNTCP/hMRP2

MDCK II cells (Madin-Darby canine kidney cells, strain II) were cultured in Dulbecco's modified essential medium supplemented with 10% fetal calf serum at 37° C., 5% CO₂, and 95% humidity. The MDCKII-hNTCP/hMRP2 cell line (DSM ACC2644) was generated as follows: MDCK II cells were first transfected with the mammalian expression plasmid pcDNA3.1zeo(+)-hMRP2 using the FuGene6 transfection reagent as described by the manufacturer (Roche). Transfected cells were selected with 1000 μg/ml Zeocin. During selection the tissue culture medium was additionally supplemented with 100 units/ml penicillin G and 100 μg/ml streptomycin sulfate. The resulting cell clones were screened in the immunoblot for recombinant human MRP2 expression followed by analysis of the proper subcellular localization of recombinant human MRP2 using Confocal Laser Scanning Microscopy. The cell clone with the highest expression of human MRP2 and concomitant localization of the recombinant MRP2 at the apical membrane was chosen for FuGene6 transfection with the mammalian expression plasmid pcDNA3.1neo(+)-hNTCP. Transfected cells were selected with 800 μg/ml Geneticin and 1000 μg/ml Zeocin. During selection the tissue culture medium was additionally supplemented with 100 units/mi penicillin G and 100 μg/ml streptomycin sulfate. Double-transfected cell lines were again screened in the immunoblot for the expression of both, recombinant human MRP2 and recombinant human NTCP, followed by analysis of the proper subcellular localization of both transgenes by Confocal Laser Scanning Microscopy. The cell clone with the highest expression and proper subcellular localization of both, human MRP2 (apical membrane) and human NTCP (basolateral membrane) was chosen for further examinations.

### Cell Line Generation: MDCKII-hNTCP

MDCK II cells (Madin-Darby canine kidney cells, strain II) were cultured in Dulbecco's modified essential medium supplemented with 10% fetal calf serum at 37° C., 5% CO₂, and 95% humidity. For generation of the MDCKII-hNTCP cell line MDCKII cells were transfected with the mammalian expression plasmid pcDNA3.1neo(+)-hNTCP using the FuGene6 transfection reagent as described by the manufacturer (Roche). Transfected cells were selected with 800 μg/ml Geneticin. During selection the tissue culture medium was additionally supplemented with 100 units/ml penicillin G and 100 μg/ml streptomycin sulfate. The resulting cell clones were screened in the immunoblot for recombinant human NTCP expression followed by functional analysis of the cell clones with the highest human NTCP expression (measurement of taurocholic acid uptake in MDCKII-hNTCP cells compared with parental MDCKII cells).

Chemicals: [³H(G)]-Bromosulfophthalein (BSP) (0.2 TBq/mmol) was purchased from Hartmann Analytic (Braunschweig, Germany). [2,4-³H(N)]-Cholic acid (0.7 TBq/mmol), [Carboxyl-14C]-Chenodeoxycholic acid (1.9 GBq/mmol) and [Glycine-2-³H]-Glycocholic acid (0.15 TBq/mmol) were obtained from Biotrend Chemikalien (Köln, Germany). [³H(G)]-Taurocholic acid (0.1 TBq/mmol) was purchased from PerkinElmer Life Sciences. Zeocin, geneticin, penicillin G, and streptomycin sulfate were obtained from Invitrogen. Additional chemicals of analytical purity were obtained from Sigma and Merck (Darmstadt, Germany)

### Transport Assays

Cells were grown at confluency (1×10⁶ cells /insert) on polyester membrane inserts (pore size 0.4 μM, insert diameter 24 mm, Costar) in 6-well plates for 6 days. Overexpression of transgenes was induced by addition of 10mM sodium butyrate to the growth medium for 24 h. The next day cells were washed once with transport assay buffer (142mM NaCl, 5mM KCl, 5mM Glucose, 1.5mM CaCl₂, 1.2mM MgSO₄, 1mM KH₂PO₄, 12.5 mM HEPES, pH 7.3) followed by addition of [³H]- or [¹⁴C]-labeled compounds either to the apical (1.5 ml) or to the basolateral (2.5 ml) compartments. The opposite compartments were filled with transport assay buffer. After the indicated incubation periods (37° C.) a sample was taken from every compartment and measured by scintillation counting (Wallac, Winspectral 1414 Liquid Scintillation Counter). The transcellular transport in percent of input was calculated as follows:

Transport from the apical to the basolateral compartment:
Transport_{A-B [%]=}100×(Radioactivity₍ₜ₁₎basolateral [dpm]×2.5 [ml])/(Radioactivity₍ₜ₀₎apical [dpm]×1.5 [ml])

Transport from the basolateral to the apical compartment:
Transport_{B-A}[%]=100×(Radioactivity₍ₜ₁₎apical [dpm]×1.5 [ml])/(Radioactivity₍ₜ₀₎basolateral [dpm]'2.5 [ml])

The following deposits of biological material were made in context of the present invention with DSMZ—Deutsche Sammlung von Mikroorganismen und Zellkulturen GmbH, Mascheroder Weg 1b; D-38124 Braunschweig:

DSM ACCZ643: Double transfected MDCKII cells harboring a vector for expressing human NTCP (Seq ID No. 4) and a vector for expressing human BSEP (Seq ID No. 5).

DSM ACC2644: Double transfected MDCKII cells harboring a vector for expressing human NTCP (Seq ID No. 4) and a vector for expressing human MRP2 (Seq ID No. 6).

### DESCRIPTION OF THE FIGURES

FIG. 1:

General Assay Design

The cells are grown as a polarized and tight monolayers in filter inserts on a porous membrane (polyester membrane, pore size 0.4 μM, 6-well) thereby separating a basolateral from an apical compartment. FIG. 1 is depicting an example of an filter-insert carrying a monolayer of cells.

FIG. 2:

Assay Design MDCK II-hNTCP/hBSEP

Schematic representation of MDCK II cells (A) and MDCK II cells transfected with human BSEP (B), or human NTCP (C), or both human NTCP and human BSEP (D). Human NTCP is localized at the basolateral membrane whereas human BSEP is localized at the apical membrane. The mono-transfected cell lines MDCKII-hBSEP and MDCKII-hNTCP serve as controls.

FIG. 3:

Assay Design MDCK II-hNTCP/hMRP2

Schematic representation of MDCK II cells (A) and MDCK II cells transfected with human MRP2 (B), or human NTCP (C), or both human NTCP and human MRP2 (D). Human NTCP is localized at the basolateral membrane whereas human MRP2 is localized at the apical membrane. The mono-transfected cell lines MDCKII-hMRP2 and MDCKII-hNTCP serve as controls.

FIG. 4:

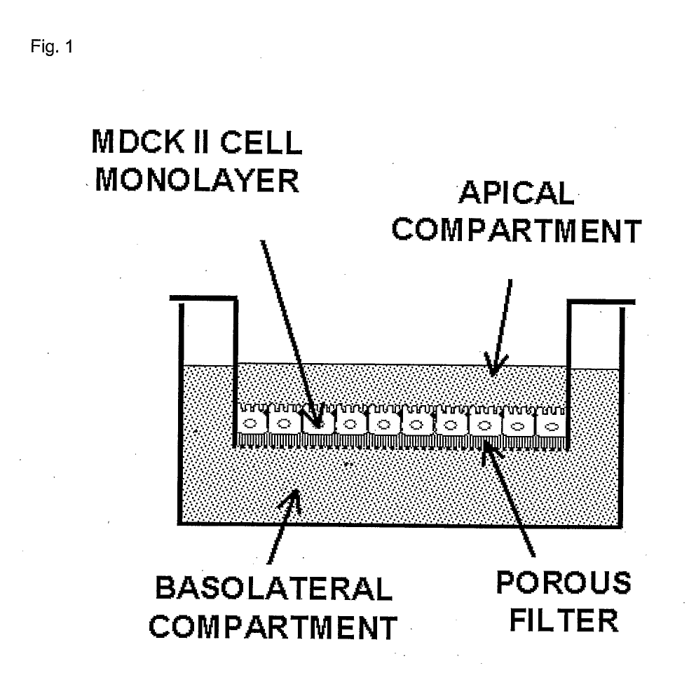

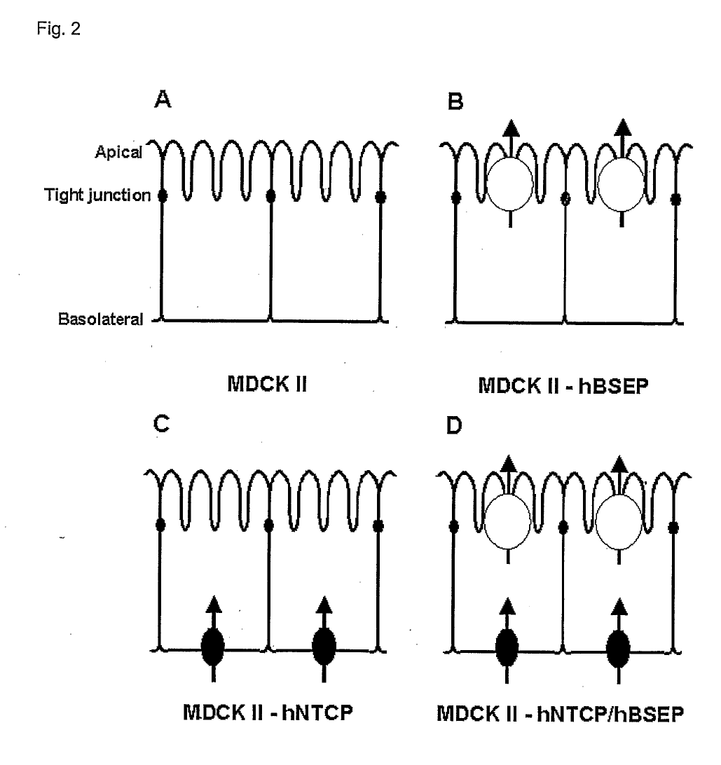

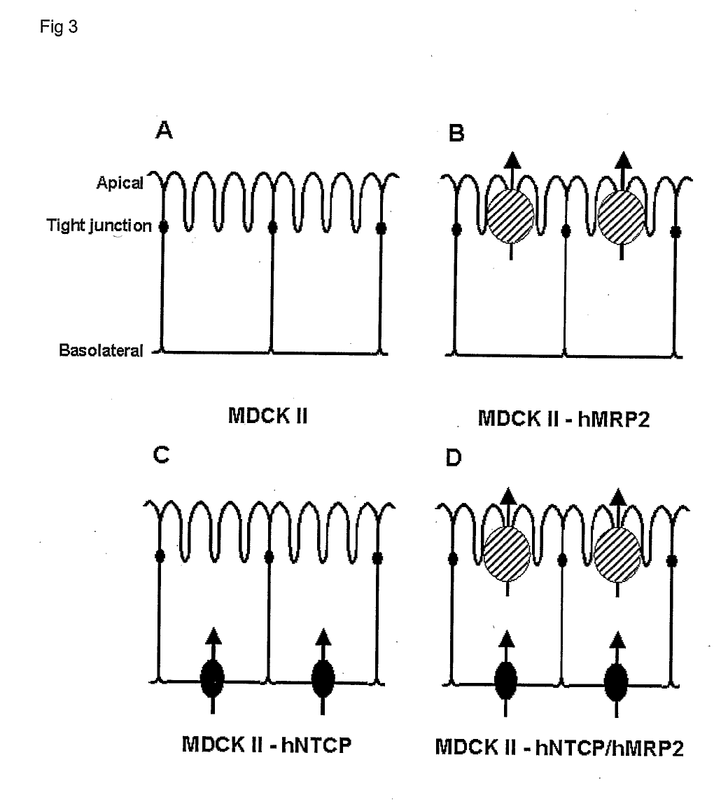

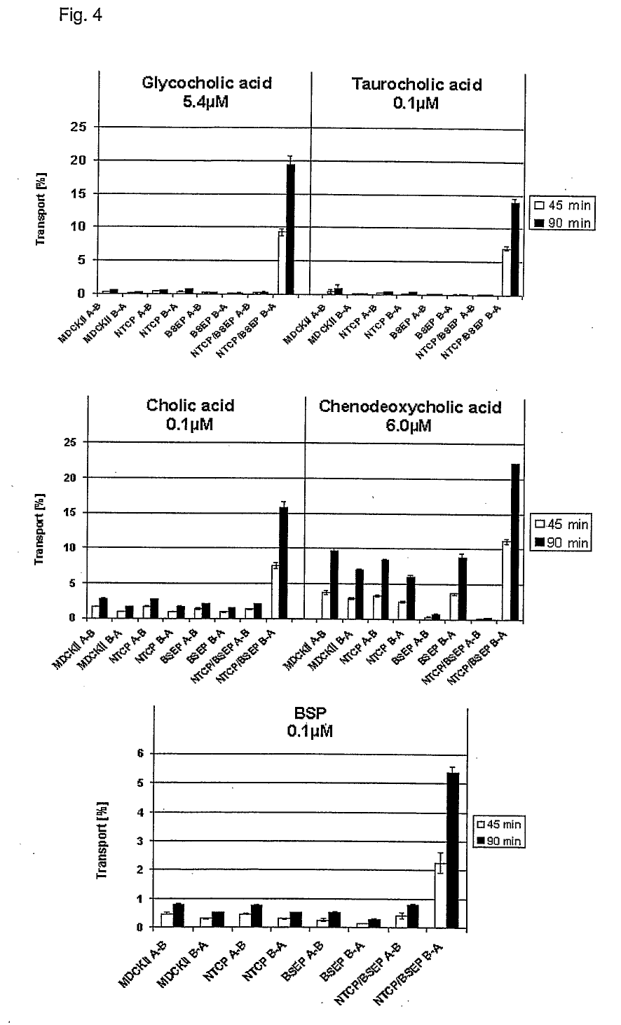

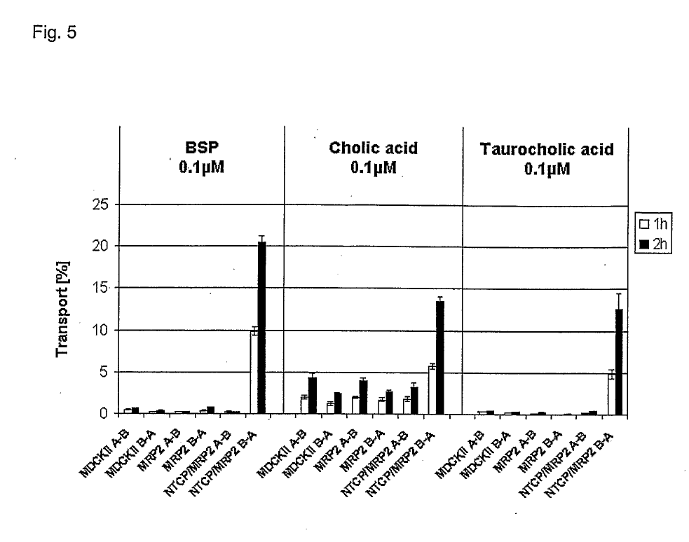

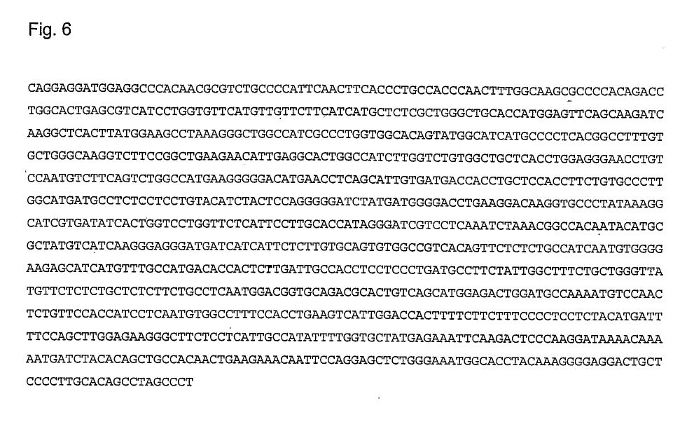

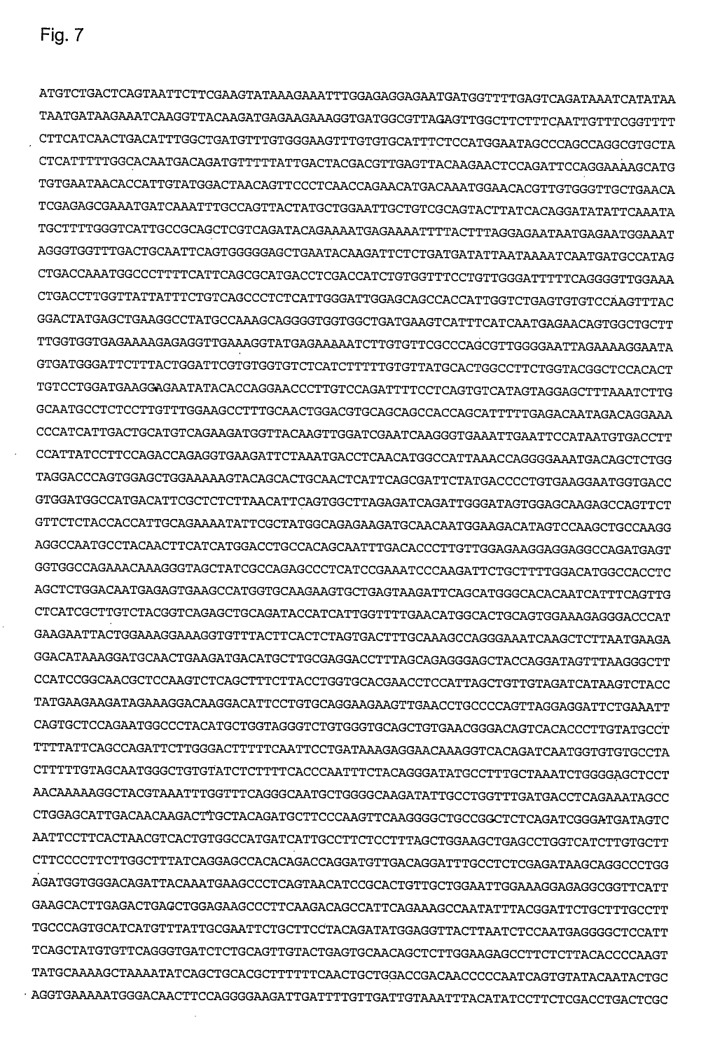

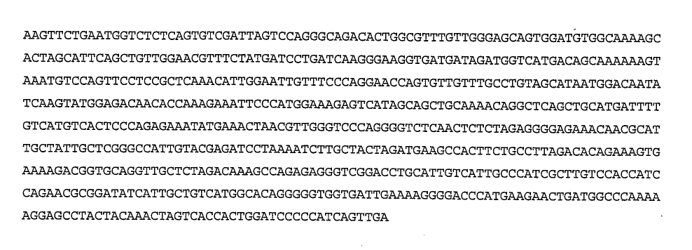

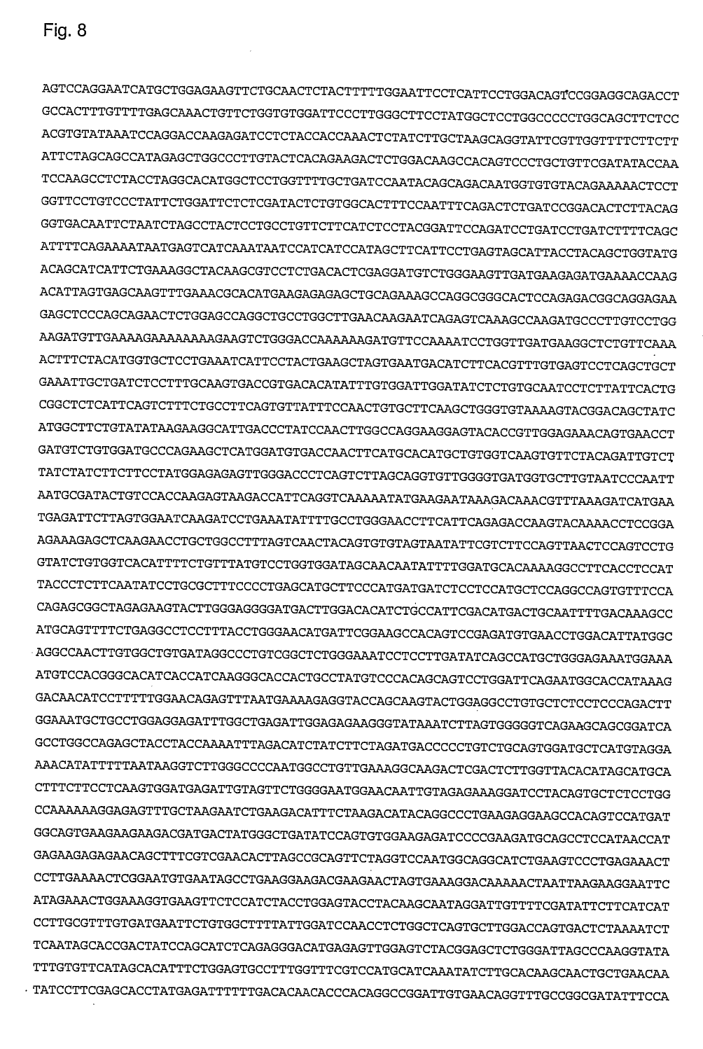

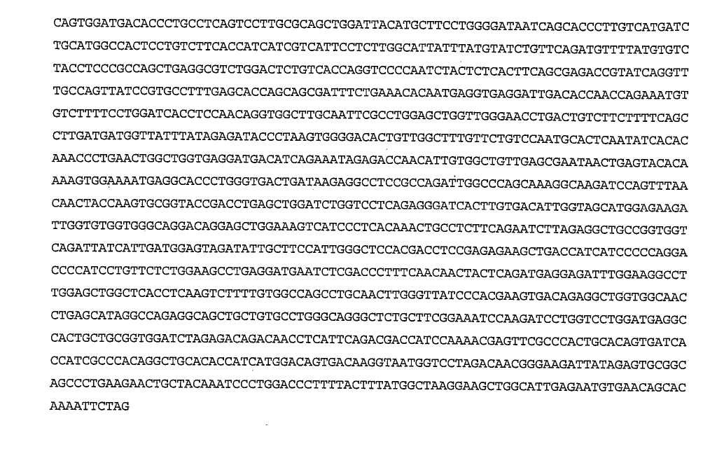

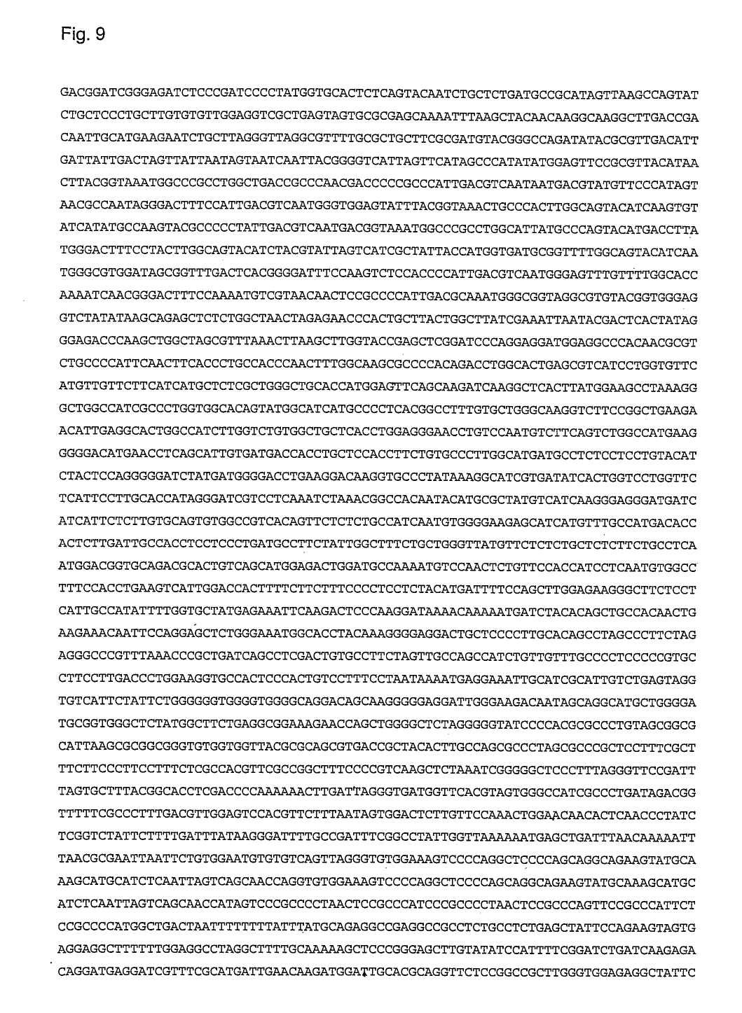

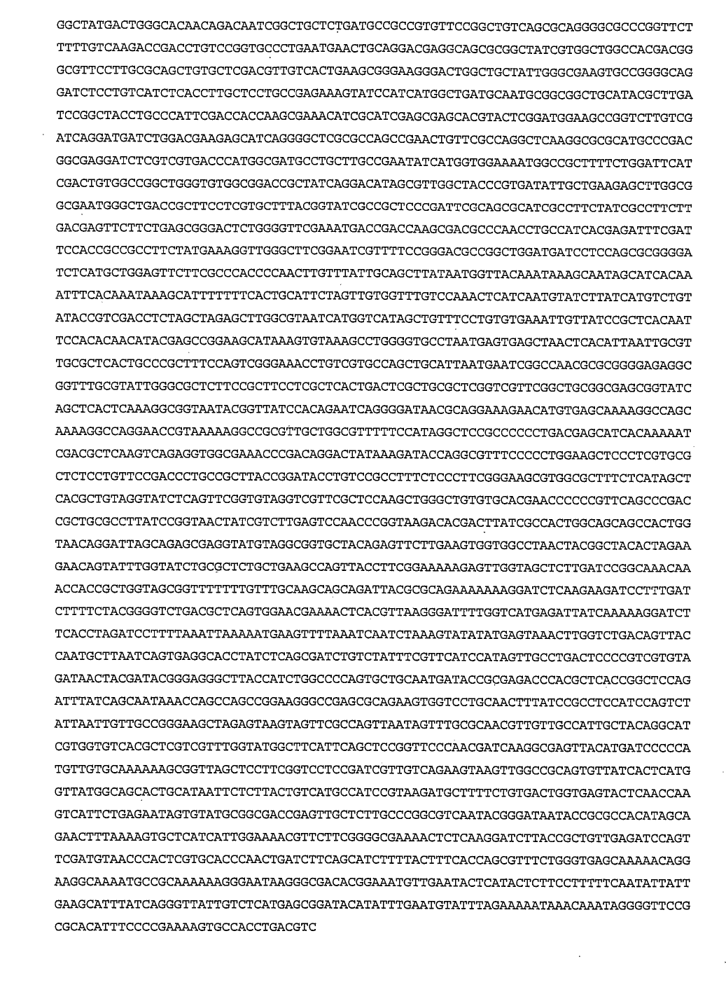

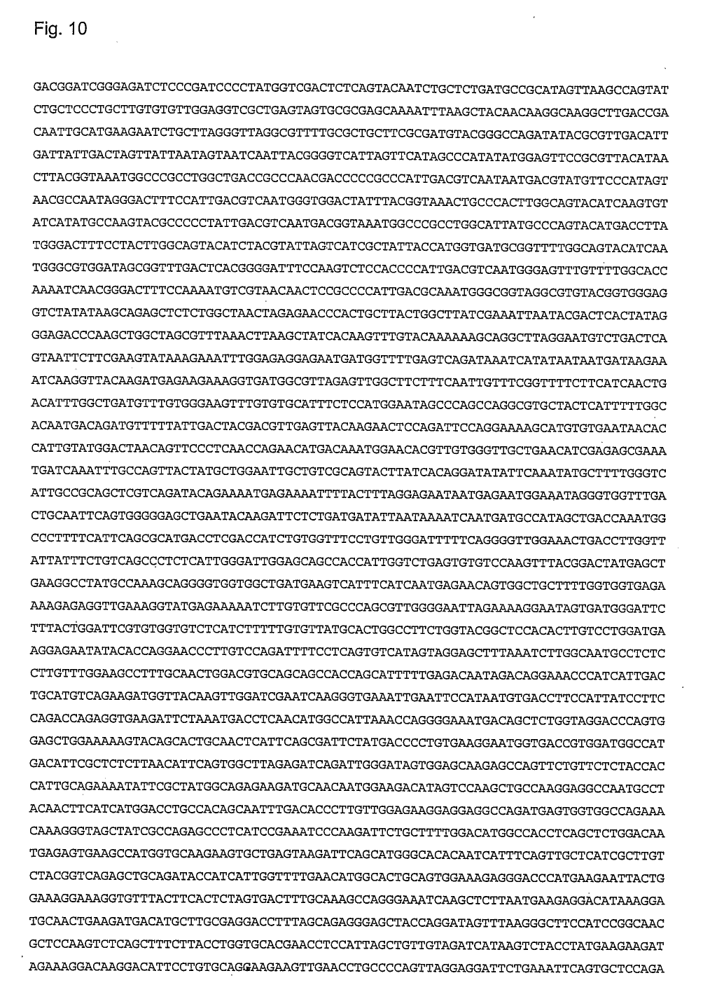

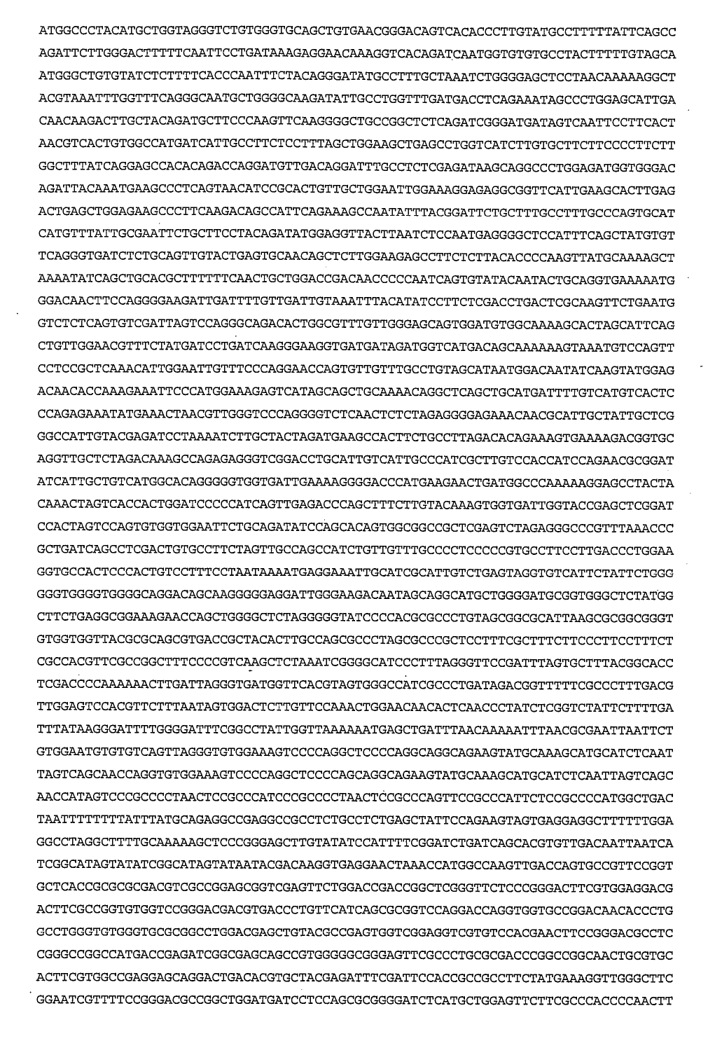

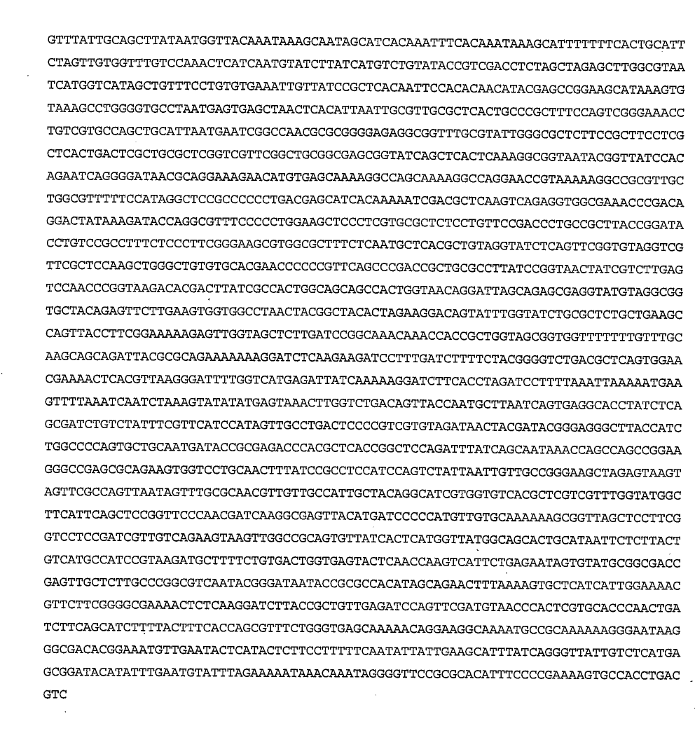

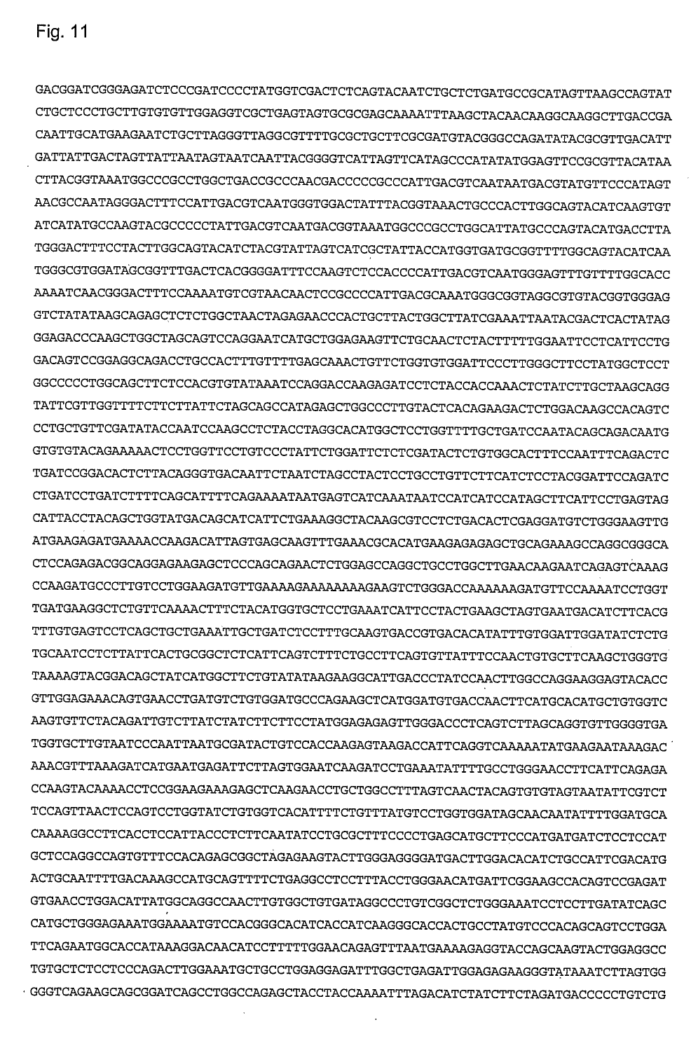

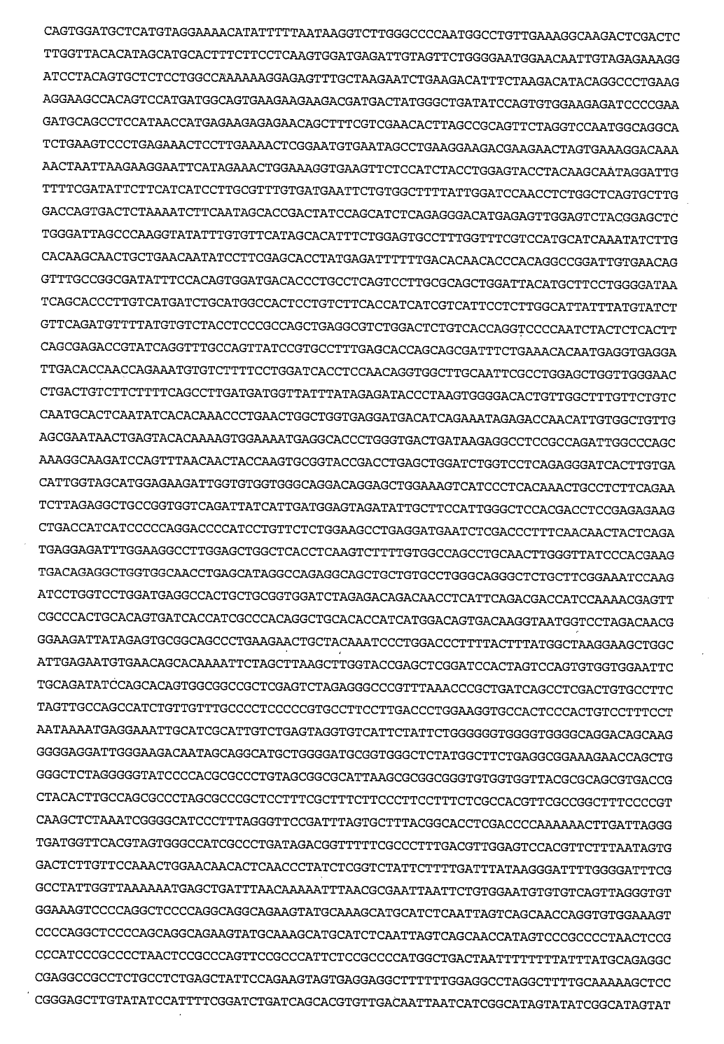

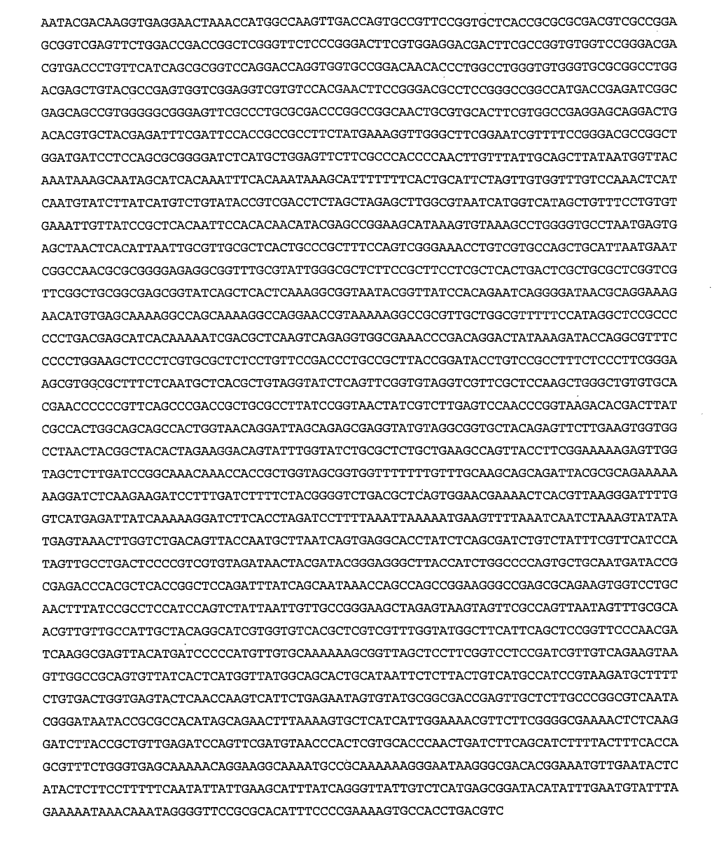

Vectorial Transport of [³H]Glycocholic Acid, [³H]Taurocholic Acid, [³H]Cholic Acid, [¹⁴C]Chenodeoxycholic Acid And [³H]BSP (Bromosulfophthalein) In MDCKII-hNTCP/hBSEP Monolayers

MDCKII, MDCKII-hNTCP, MDCKII-hMRP2 and MDCKII-hNTCP/hMRP2 cells were grown on filter inserts for 6 days (1×10⁶ cells/well). Transgene expression was induced by addition of 10mM sodium butyrate for 24 h. The indicated amount of each compound was given separately either to the basolateral or to the apical compartment. After 45 and 90 minutes an aliquot was taken from the opposite compartment and analysed by liquid scintillization counting. A-B indicates addition of compound to the apical compartment and sampling from the basolateral compartment (vectorial transport from the apical to the basolateral compartment). B-A indicates addition of compound to the basolateral compartment and sampling from the apical compartment (vectorial transport from the basolateral to the apical compartment). Data represent means±SD (n=3).

FIG. 5:

Vectorial Transport of [³H]BSP (Bromosulfophthalein), [³H]Cholic Acid And [³H]Taurocholic Acid In MDCKII-hNTCP/hMRP2 Monolayers

MDCKII, MDCKII-hMRP2 and MDCKII-hNTCP/hMRP2 cells were grown on filter inserts for 6 days (1×10⁶ cells/well). Transgene expression was induced by addition of 10mM sodium butyrate for 24 h. 100nM of each compound was given separately either to the basolateral or to the apical compartment. After one and two hours an aliquot was taken from the opposite compartment and analysed by liquid scintillization counting. A-B indicates addition of compound to the apical compartment and sampling from the basolateral compartment (vectorial transport from the apical to the basolateral compartment). B-A indicates addition of compound to the basolateral compartment and sampling from the apical compartment (vectorial transport from the basolateral to the apical compartment). Data represent means±SD (n=3).

FIG. 6:

Human NTCP (Slc10A1), cDNA sequence; Length: 1061 bases; the depicted sequence is corresponding to Seq ID No. 1.

FIG. 7:

Human BSEP (ABCB11), cDNA sequence; Length: 3966 bases; the depicted sequence is corresponding to Seq ID No. 2.

FIG. 8:

Human MRP2 (ABCC2), cDNA sequence; Length: 4650 bases; the depicted sequence is corresponding to Seq ID No. 3.

FIG. 9:

pcDNA3.1neo(+)-hNTCP, complete sequence; Length: 6433 bases; the depicted sequence is corresponding to Seq ID No. 4:

The human Sodium-dependent Tauchocholate Cotransporting Protein (hNTCP, SLC10A1) cDNA was cloned into the MCS of pcDNA3.1neo(+) (Invitrogen) via BamH I and Xba I. Human NTCP cDNA was PCR-amplified with gene-specific primers from a human liver cDNA library (Clontech). The cloned cDNA corresponds to GenBank Accession #L21893 from position 76-1136 (coding sequence: position 83-1132). Exceptions: position 616 c→t (no amino acid change) and position 774 t→c (amino acid change F→S).

FIG. 10:

pcDNA3.1zeo(+)-hBSEP, complete sequence; Length: 9043 bases; the depicted sequence is corresponding to Seq ID No. 5:

The human Bile Salt Export Pump (hBSEP, ABCB11) CDNA was cloned by using the Gateway Technology (Invitrogen). Human BSEP cDNA was PCR-amplified with attB-modified gene-specific primers from a human fetal liver cDNA library (Clontech) and cloned via pDONR221 into the pcDNA3.1zeo(+) vector (Invitrogen, modified to a Gateway destination vector). The cloned cDNA corresponds to GenBank Accession #AF136523 from position 128-4093 (coding sequence: position 128-4093). Exception: position 3211 G→A (no amino acid change).

FIG. 11:

pcDNA3.1zeo(+)-hMRP2, complete sequence; Length: 9658 bases; the depicted sequence is corresponding to Seq ID No. 6:

The human Multidrug-resistance Related Protein 2 (MRP2, ABCC2) cDNA was cloned into the MCS of pcDNA3.1zeo(+) (Invitrogen) via Nhe I and AfI II. Human MRP2 cDNA was PCR-amplified with gene-specific primers from a HepG2 cDNA library. The cloned cDNA corresponds to GenBank Accession #X96395 from position 26-4675 (coding sequence: position 38-4675). Exception: position 4009 C→T (no amino acid change).

### Description of Seq IDs

Seq ID No. 1:

Human NTCP (Slc10A1), cDNA sequence; Length: 1061 bases; Seq ID No. 1 is disclosed in FIG. 6.

Seq ID No. 2:

Human BSEP (ABCB11), cDNA sequence; Length: 3966 bases; Seq ID No. 2 is disclosed in FIG. 7.

Seq ID No. 3:

Human MRP2 (ABCC2), cDNA sequence; Length: 4650 bases; Seq ID No. 3 is disclosed in FIG. 8.

Seq ID No. 4:

pcDNA3.1neo(+)-hNTCP, complete sequence; Length: 6433 bases; Seq ID No. 4 is disclosed in FIG. 9.

Seq ID No. 5:

pcDNA3.1zeo(+)-hBSEP, complete sequence; Length: 9043 bases; Seq ID No. 5 is disclosed in FIG. 10.

Seq ID No. 6:

pcDNA3.1zeo(+)-hMRP2, complete sequence; Length: 9658 bases; Seq ID No. 6 is disclosed in FIG. 11.

### REFERENCES

1. Louvard D. (1980) Apical membrane aminopeptidase appears at site of cell-cell contact in cultured kidney epithelial cells. Proc. Natl. Acad. Sci. USA 77(7): 4132-4136

2. Fuller S, von Bonsdorff C-H, and Simons K (1984) Vesicular Stomatitis Virus infects and matures only through the basolateral surface of the polarized epithelial cell line, MDCK. Cell 38: 65-77

1. A mammalian cell comprising:
a] a first side comprising a functional hNTCP protein; and
b] a second side comprising a functional HBSEP protein
wherein the first side is distinguished from the second side by localization at opposite ends of said cell.

2. The mammalian cell according to claim 1 wherein the first side is the basolateral side and the second side is the apical side.

3. (canceled)

4. The mammalian cell according to claim 1 wherein the cell is an epithelial cell of an organ or a system wherein the organ or the system is selected from the group consisting of kidney, bowels system, liver and blood/brain barrier.

5. The mammalian cell according to claim 1 wherein the cell is immortalized.

6. The mammalian cell according to claim 1 wherein the cell is a recombinant cell.

7. The mammalian cell according to claim 1 wherein the cell is a LLC-PK1 cell comprising one or more vector(s) for expressing the hNTCP protein and the hBSEP protein.

8. The mammalian cell according to claim 1 wherein the cell is a MDCKII cell comprising one or more vector(s) for expressing the hNTCP protein and the HBSEP protein.

9. The mammalian cell according to claim 1 wherein the cell is deposit DSM ACC2643 or progeny thereof.

10. A method of manufacturing a mammalian cell according to claim 1 comprising:
a] providing a mammalian cell;
b] providing a vector comprising the coding sequence of hNTCP;
c] providing a vector comprising the coding sequence of HBSEP;
d] transforming the mammalian cell from a] by a vector from b] and by a vector from c] either simultaneously or consecutively;
e] identifying and propagating a double transfectant cell from d].

11. The method of claim 10 wherein the mammalian cell from a] is an epithelial cell of an organ or a system wherein the organ or the system is selected from the group consisting of kidney, bowels system, liver and blood/brain barrier.

12. The method of claim 10 wherein the mammalian cell from a] is immortalized.

13. The method of claim 10 wherein the vector from b] is a polynucleotide according to FIG. 9 (Seq ID No. 4).

14. The method of claim 10 wherein the vector from c] is a polynucleotide according to FIG. 10 (Seq ID No. 5).

15. The method of claim 10 wherein the mammalian cell comprises deposited cell DSM ACC2643 or the progeny thereof.

16. A monolayer of cells comprising at least two cells according to claim 1.

17. The monolayer of cells according to claim 16 wherein the monolayer of cells occupy part or whole of a solid surface.

18. The solid surface according to claim 17 wherein the solid surface comprises a plastic.

19. The solid surface as claimed in according to claim 17 wherein the solid surface comprises a petri dish.

20. The solid surface as claimed in according to claim 17 wherein the solid surface comprises a filter membrane.

21. A petri dish carrying a monolayer of cells according to claim 20.

22. The filter membrane according to claim 20 wherein the filter membrane comprises a filter-insert.

23. The filter-insert according to claim 22 wherein the filter membrane comprises polycarbonate or polyester.

24. The filter-insert according to claim 22 wherein the filter membrane has a pore size of 0.4 μm.

25. A method for determining a pharmacological profile comprising:
a] providing a monolayer of cells according to claim 16;
b] adding a labeled compound to a first compartment of the monolayer;
c] incubating the labeled compound with the monolayer;
d] measuring the amount of the labeled compound in the first compartment of the monolayer and in a second compartment of the monolayer; and
e] comparing the amount of the labeled compound in the first compartment to the second compartment to determine a difference
thereby identifying the pharmacological profile wherein the pharmacological profile is selected from the group consisting of hepatobiliary elimination, renal excretion, brain resorption and intestinal resorption.

26. The method according to claim 25 wherein the mammalian cell forms part of a monolayer on a solid surface wherein the solid surface is selected from the group consisting of a petri dish, a filter membrane and a filter-insert.

27. A mammalian cell comprising:
a] a first side comprising a functional HNTCP protein; and
b] a second side comprising a functional HBSEP protein
wherein the first side is distinguished from the second side by localization at opposite ends of said cell.

28. The mammalian cell according to claim 27 wherein the first side is the basolateral side and the second side is the apical side.

29. (canceled)

30. The mammalian cell according to claim 27 wherein the cell is an epithelial cell of an organ or a system wherein the organ or the system is selected from the group consisting kidney, bowels system, liver and blood/brain barrier.

31. The mammalian cell according to claim 27 wherein the cell is immortalized.

32. The mammalian cell according to claim 27 wherein the cell is a recombinant cell.

33. The mammalian cell according to claim 27 wherein the cell is a LLC-PKl cell comprising one or more vector(s) for expressing the hNTCP protein and the hMRP2 protein.

34. The mammalian cell according to claim 27 wherein the cell is a MDCKII cell comprising one or more vector(s) for expressing the hNTCP protein and the hMRP2 protein.

35. The mammalian cell according to claim 27 wherein the cell is deposited as DSM ACC2644 or progeny thereof.

36. A method of manufacturing a mammalian cell according to claim 27 comprising:
a] providing a mammalian cell is provided;
b] providing a vector is provided en comp rising the coding sequence of hNTPC;
c] providing a vector is provided encompassing comprising the coding sequence of hMRP2;
d] transforming the mammalian cell from a] by a vector from b] and by a vector from c] either simultaneously or consecutively;
e] identifying and propagating a double transfectant cell from d].

37. The method of claim 36 wherein the mammalian cell from a] is an epithelial cell of an organ or a system wherein the organ or the system is selected from the group consisting of kidney, bowels system, liver and blood/brain barrier.

38. The method of claim 36 wherein the mammalian cell from a] is immortalized.

39. The method of claim 36 wherein the vector from b] is a polynucleotide according to FIG. 9 (Seq ID No. 4).

40. The method of claim 36 wherein the the vector from c] is a polynucleotide according to FIG. 11 (Seq ID No. 6).

41. The method of claim 36 wherein the mammalian cell comprises deposited cell DSM ACC2644 or the progeny thereof.

42. A monolayer of cells comprising at least two cells according to claim 36.

43. The monolayer of cells according to claim 42 wherein the monolayer of cells occupy part or whole of a solid surface.

44. The solid surface according to claim 43 wherein the solid surface comprises a plastic.

45. The solid surface according to claim 43 wherein the solid surface comprises a petri dish.

46. The solid surface according to claim 43 wherein the solid surface comprises a filter membrane.

47. A petri dish carrying a monolayer of cells according to claim 46.

48. The filter membrane according to claim 46 wherein the filter membrane comprises a filter-insert.

49. The filter-insert according to claim 48 wherein the filter membrane comprises polycarbonate and/or polyester.

50. The filter-insert according to claim 48 wherein the filter membrane has a pore size of 0.4 μm.

51. A method for determining a pharmacological profile comprising:
a] providing a monolayer of cells according to claim 42;
b] adding a labeled compound to a first compartment of the monolayer;
c] incubating the labeled compound with the monolayer:
d] measuring the amount of the labeled compound in the first compartment of the monolayer and in a second compartment of the monolayer; and
e] comparing the amount of the labeled compound in the first compartment to the second compartment to determine a difference
thereby identifying the pharmacological profile wherein the pharmacological profile is selected from the group consisting of hepatobiliary elimination, renal excretion, brain resorption and intestinal resorption.

52. The method according to claim 51 wherein the mammalian cell forms part of a monolayer on a solid surface wherein the solid surface is selected form the group consisting of a petri dish, a filter membrane and a filter-insert.
